(19) United States
(12) Patent Application Publication
(10) Pub. No.: US 2007/0237803 A1
(43) Pub. Date: Oct. 11, 2007

(54) Biodegradable Biocompatible Amphiphilic Copolymers for Coating and Manufacturing Medical Devices

(75) Inventors: Peiwen Cheng, Santa Rosa, CA (US); Ya Guo, Rohnert Park, CA (US); Mingfei Chen, Santa Rosa, CA (US); Kishore Udipi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/619,122

(22) Filed: Jan. 2, 2007

Related U.S. Application Data
(60) Provisional application No. 60/744,629, filed on Apr. 11, 2006.

(51) Int. Cl. A61F 2/00; C08F 20/00

(52) U.S. Cl. 424/426; 525/438

(57) ABSTRACT

Disclosed in the present invention are biodegradable biocompatible amphiphilic copolymers for coating and manufacturing medical devices. The properties of the polymers in the present invention are fine tuned for optimal performance depending on the medical purpose. Moreover, the polymers of the present invention retain and release bioactive drugs in a controlled manner.

### RELATED APPLICATIONS

This application claims the benefit of U.S. Provisional Patent Application 60/744,629 filed Apr. 11, 2006.

### FIELD OF THE INVENTION

The present invention relates to drug-eluting biodegradable biocompatible amphiphilic copolymers suitable for coating and manufacturing medical devices.

### BACKGROUND OF THE INVENTION

The role of polymers in the medical industry is rapidly growing. Polymers have seen use in surgical adhesives, sutures, tissue scaffolds, heart valves, vascular grafts and other medical and surgical products. One area that has seen noteworthy growth is implantable medical devices. Biocompatible polymers are particularly useful for manufacturing and coating implantable medical devices. Biodegradable biocompatible polymers suitable for coating and constructing medical devices generally include polyesters such as polylactide, polyglycolide, polycaprolactone, their copolymers or cellulose derivatives, collagen derivatives.

Properties advantageous for polymers used for medical devices include biocompatibility and, in some applications, biodegradability. The merits of biocompatible polymers include decreased inflammatory response, decreased immunological response and decreased post-surgical healing times. Biodegradability is advantageous for implanted medical devices since, in certain circumstances, the medical device would otherwise require a second surgery to remove the device after a period of time. Polymers can be rendered biodegradable biocompatible by modifying the monomer composition. In one example, an adhesive composition for surgical use was made biodegradable by copolymerizing caprolactone, ethylene glycol and DL lactic acid (see, for example, U.S. Pat. No. 6,316,523).

Additionally, polymers are used to deliver drugs from an implantable medical device made of another material wherein the polymer is coated on at least one surface of the medical device, thereby allowing for controlled drug release directly to the implantation site. Hydrophobic polymers including polylactic acid, polyglycolic acid and polycaprolactone are generally compatible with hydrophobic drugs. Hydrophilic polymers conversely are more compatible with hydrophilic drugs. Polymer-drug incompatibility hurdles are overcome by using amphiphilic polymers. Amphiphilic, as used herein, refers to the polymer having both hydrophobic and hydrophilic properties. In one example, biodegradable biocompatible amphiphilic polymers are provided with hydrophilic groups containing poly-ionic organic moieties and the hydrophobic portion of the polymer contains a steroid, e.g. cholesterol coupled to a poly-lactide (see U.S. Pat. No. 5,932,539).

Drug-releasing amphiphilic polymers can be formulated into microspheres that contain the drugs. For example, retinoic acid has been encapsulated in a microsphere made of an amphiphilic polymer (see U.S. Pat. No. 6,841,617). The hydrophilic portions of the polymer are made of polyethylene glycol (PEG) while polylactic acid forms the hydrophobic portion of the polymer. This design provides a hydrophilic portion of the polymer on the outside of the microsphere which is exposed to the aqueous environment while the hydrophobic portion is on the inside of the microsphere and is not exposed to the aqueous environment and thus the microsphere encapsulates the retinoic acid.

Implanted medical devices that are coated with biodegradable biocompatible polymers offer substantial benefits to the patient. Reduced inflammation and immunological responses promote faster post-implantation healing times in contrast to uncoated medical devices. Polymer-coated vascular stents, for example, may encourage endothelial cell proliferation and therefore integration of the stent into the vessel wall. Loading the coating polymers with appropriate drugs is also advantageous in preventing undesired biological responses. For example, an implanted polylactic acid polymer loaded with hirudin and prostacyclin does not generate thrombosis, a major cause of post-surgical complications (Eckhard et al, Circulation, 2000, pp 1453-1458).

There is a need for improved polymeric materials suitable for implantation. Implantable medical devices containing such polymers should possess properties such as reducing the negative effects seen with implanted medical devices. The implantable polymeric materials should be able to deliver hydrophilic and hydrophobic drugs, effectively coat the medical device and be biodegradable.

### SUMMARY OF THE INVENTION

The present invention relates to biodegradable biocompatible amphiphilic polymers suitable for forming and coating medical devices and controlling in situ drug release. The polymers of the present invention have polyester and polyether backbones and are comprised of monomers including, but not limited to, ε-caprolactone, polyethylene glycol (PEG), trimethylene carbonate, lactide, and their derivatives. Structural integrity and mechanical durability are provided through the use of lactide. Elasticity is provided by caprolactone and trimethylene carbonate while PEG provides a hydrophilic character. Therefore the amphiphilic polymers of the present invention are capable of delivering both hydrophobic and hydrophilic drugs to a treatment site. Furthermore, the amphiphilic polymers of the present invention are biodegradable. Varying the monomer ratios allows the practitioner to fine tune, or modify, the properties of the polymer to control physical properties including drug elution rates.

The properties of biodegradable biocompatible amphiphilic polymers are a result of the monomers used and the reaction conditions employed in their synthesis including, but not limited to, temperature, solvent choice, reaction time and catalyst choice.

The polymers made in accordance with the present invention are also suitable for manufacturing implantable medical devices. In one embodiment of the present invention, a medical device is manufactured from a biodegradable biocompatible amphiphilic polymer of the present invention. In another embodiment, the biodegradable biocompatible amphiphilic polymer is provided as a coating on a medical device. In yet another embodiment, a drug is provided in the biodegradable biocompatible amphiphilic polymer medical device or coating.

Medical devices suitable for coating with the amphiphilic polymers of the present invention include, but are not limited to, vascular stents, stent grafts, urethral stents, bile duct stents, catheters, guide wires, pacemaker leads, bone screws, sutures and prosthetic heart valves. The polymers of the present invention are suitable for coating and manufacturing implantable medical devices. Medical devices which can be manufactured from the amphiphilic polymers of the present invention include, but are not limited to, vascular stents, stent grafts, urethral stents, bile duct stents, catheters, guide wires, pacemaker leads, bone screws, sutures and prosthetic heart valves.

The present invention also provides for providing biodegradable biocompatible amphiphilic polymer with properties based upon their glass transition temperatures (Tg). Drug elution from polymers depends on many factors including polymer density. The drug to be eluted, molecular nature of the polymer and Tg, among other properties. Higher Tgs, for example temperatures above 40° C., result in more brittle polymers while in most situations, when Tg below body temperature 37° C., the polymers become more pliable and elastic, if Tg around 0° C., the polymers become tacky.) In the present invention Tg can be controlled, such that the polymer elasticity and pliability can be varied as a function of temperature. The mechanical properties dictate the use of the polymers, for example, drug elution is slow from polymers that have high Tgs while faster rates of drug elution are observed with polymers possessing low Tgs.

### BRIEF DESCRIPTION OF THE DRAWINGS

FIG. 1 graphically depicts idealized first-order kinetics associated with drug release from a polymer coating.

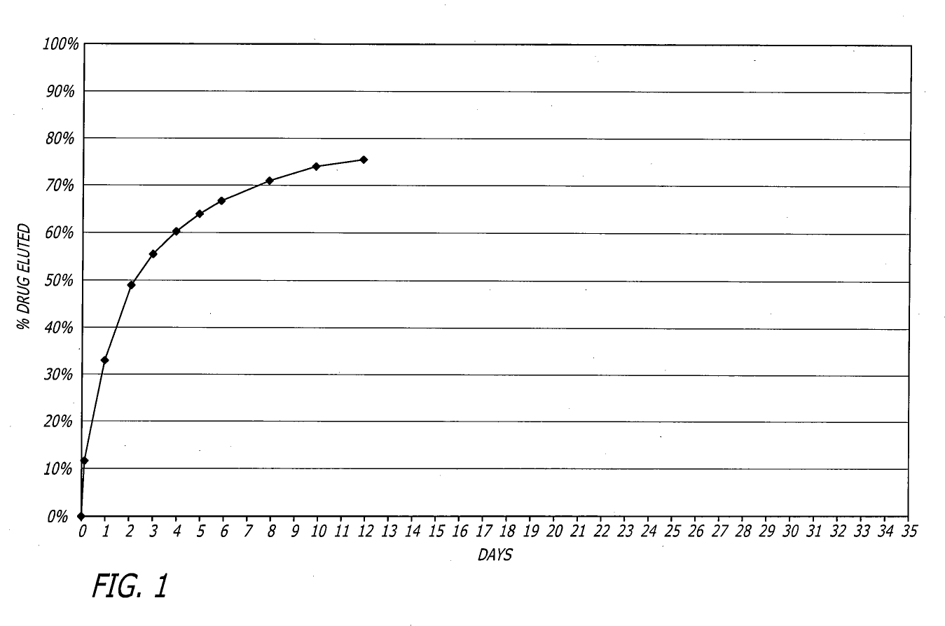

FIG. 2 graphically depicts idealized zero-order kinetics associated with drug release from a polymer coating.

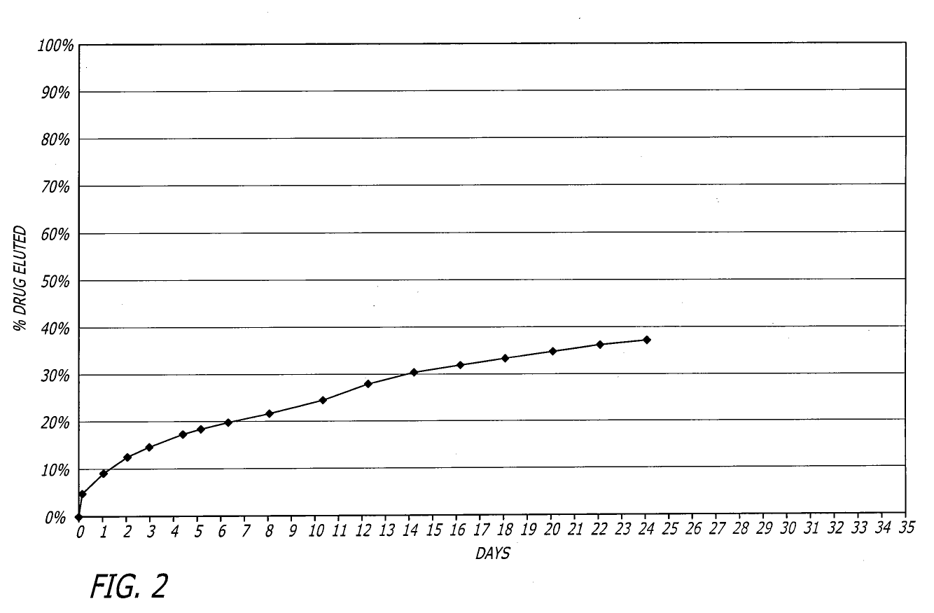

FIG. 3 graphically depicts a drug release profile of rapamycin from a 12 mm biodegradable biocompatible amphiphilic polymer coated stent.

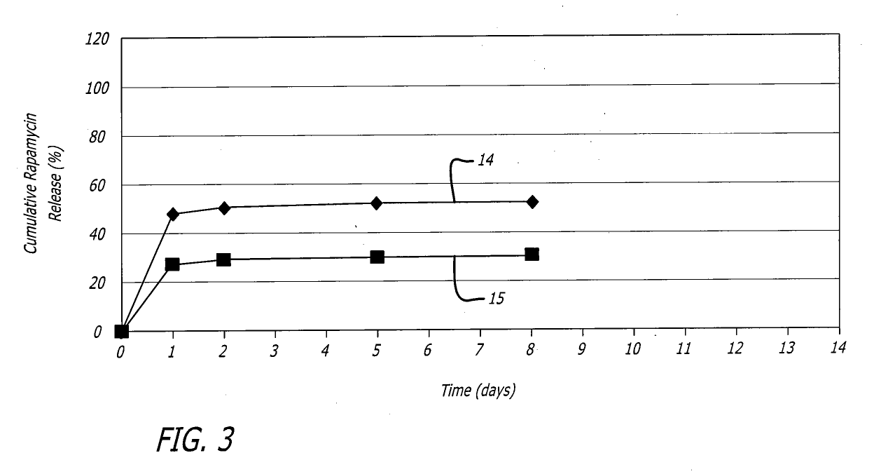

FIG. 4 depicts a table of non-limiting embodiments in accords to the teaching of the present invention. The acronyms for the monomers in FIG. 4 are as follows: PEG3400 is PEG with an average molecular weight of 3400; DLLA is DL Lactide, CL is caprolactone; DLA is D lactide; LLA is L lactide; GA is glycolide, TMC is trimethylene carbonate, t-butyl CL is 4-tert-butyl caprolactone; N-acetyl CL is N-acetyl caprolactone and is described in the definition of terms below. The feed weight ratio is the weight ratio of each monomer in polymerization. The molar feed ratio is weight ratio divided by each monomer formula weight. The final composition NMR ratio is calculated based on the specific proton ratio of each monomer that reflect their molar ratio in copolymer.

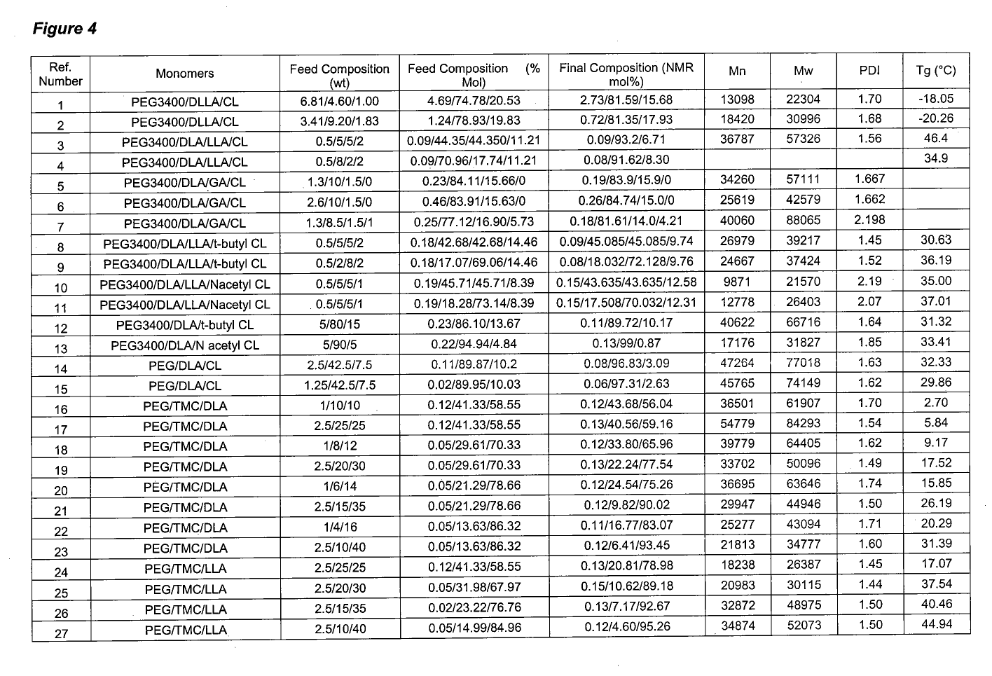

FIG. 5 graphically depicts the drug release profile of rapamycin of polymers 16, 18 and 24.

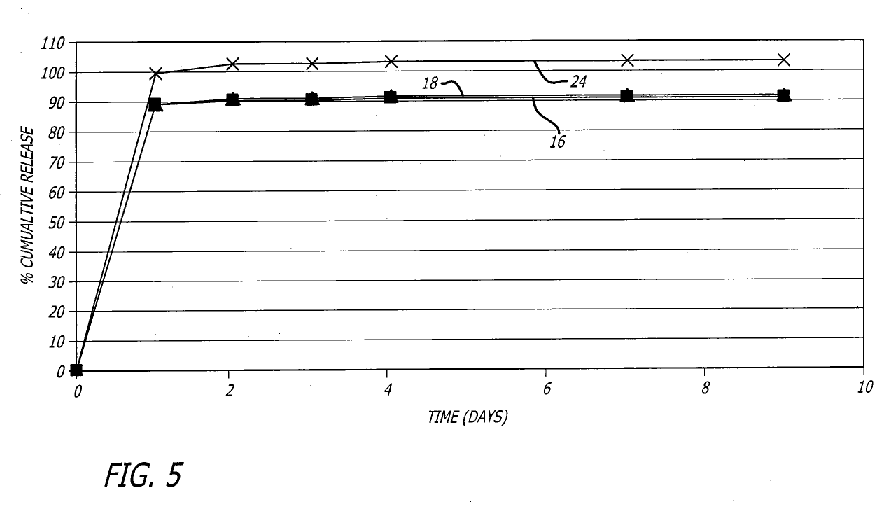

### DEFINITION OF TERMS

Prior to setting forth the invention, it may be helpful to an understanding thereof to set forth definitions of certain terms that will be used hereinafter:

Amphiphilic: As used herein, amphiphilic refers to a molecule or polymer having at least one a polar, water-soluble group and at least one a nonpolar, water-insoluble group. In simpler non limiting terms, a molecule that is soluble in both an aqueous environment and a non-aqueous environment.

Lactide: As used herein, lactide refers to 3,6-dimethyl-1,4-dioxane. More commonly lactide is also referred to herein as the heterodimer of R and S forms of lactic acid, the homodimer of the S form of lactic acid and the homodimer of the R form of lactic acid. Lactide is also depicted below in Formula 1. Lactic acid and lactide are used interchangeably herein. The term dimer is well known to those ordinarily skilled in the art.

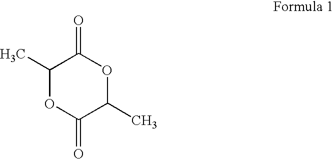

Glycolide: As used herein, glycolide refers to a chemical of the structure of Formula 2.

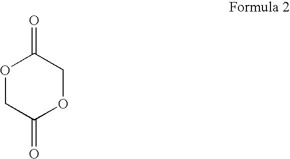

4-tert-butyl caprolactone: As used herein 4-tert-butyl caprolactone refers to a chemical of the structure of Formula 3.

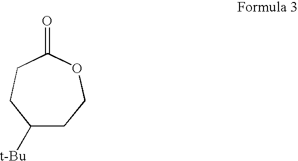

N-acetyl caprolactone: As used herein N-acetyl caprolactone refers to a chemical of the structure of Formula 4.

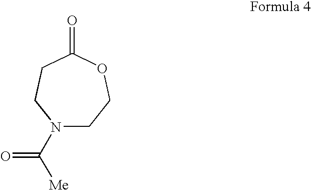

Backbone: As used here in “backbone” refers to the main chain of a polymer or copolymer of the present invention. A “polyester backbone” as used herein refers to the main chain of a biodegradable polymer comprising ester linkages. A “polyether backbone” as used herein refers to the main chain of a biodegradable polymer comprising ether linkages. An exemplary polyether is polyethylene glycol (PEG).

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however all bioresorbable polymers are considered biodegradable. The biodegradable polymers of the present invention are capable of being cleaved into biocompatible byproducts through chemical- or enzyme-catalyzed hydrolysis.

Copolymer: As used here in a “copolymer” will be defined as a macromolecule produced by the simultaneous or step-wise polymerization of two or more dissimilar units such as monomers. Copolymer shall include bipolymers (two dissimilar units), terpolymers (three dissimilar units), etc.

Compatible: As used herein “compatible” refers to a composition possing the optimum, or near optimum combination of physical, chemical, biological and drug release kinetic properties suitable for a controlled-release coating made in accordance with the teachings of the present invention. Physical characteristics include durability and elasticity/ductility, chemical characteristics include solubility and/or miscibility and biological characteristics include biocompatibility. The drug release kinetic should be either near zero-order or a combination of first and zero-order kinetics.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Drug(s): As used herein “drug” shall include any bioactive agent having a therapeutic effect in an animal. Exemplary, non limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Ductility: As used herein “ductility, or ductile” is a polymer attribute characterized by the polymer's resistance to fracture or cracking when folded, stressed or strained at operating temperatures. When used in reference to the polymer coating compostions of the present invention the normal operating temperature for the coating will be between room temperature and body temperature or approximately between 15° C. and 40° C. Polymer durability in a defined environment is often a function of its elasticity/ductility.

Functional Side Chain: As used herein “functional side chain” encompasses a first chemical constituent(s) typically capable of binding to a second chemical constituent(s), wherein the first chemical constituent modifies a chemical or physical characteristic of the second chemical constituent. Functional groups associated with the functional side chains include vinyl groups, hydroxyl groups, oxo groups, carboxyl groups, thiol groups, amino groups, phosphor groups and others known to those skilled in the art and as depicted in the present specification and claims.

Glass Transition Temperature (Tg): As used herein glass transition temperature (Tg) refers to a temperature wherein a polymer structurally transitions from a elastic pliable state to a rigid and brittle state.

Hydrophilic: As used herein in reference to the bioactive agent, the term “hydrophilic” refers to a bioactive agent that has a solubility in water of more than 200 micrograms per milliliter.

Hydrophobic: As used herein in reference to the bioactive agent the term “hydrophobic” refers to a bioactive agent that has a solubility in water of no more than 200 micrograms per milliliter.

Mₙ: As used herein Mₙ refers to number-average molecular weight. Mathematically it is represented by the following formula:

Mₙ=ΣᵢNᵢMᵢ/ΣᵢNᵢ, wherein the Nᵢ is the number of moles whose weight is Mᵢ.

M_{w}: As used herein M_{w} refers to weight average molecular weight that is the average weight that a given polymer may have. Mathematically it is represented by the following formula:

M_{w}=ΣᵢNᵢMᵢ²/ΣᵢNᵢMᵢ, wherein Nᵢ is the number of molecules whose weight is Mᵢ.

### DETAILED DESCRIPTION OF THE INVENTION

Disclosed herein are biodegradable biocompatible amphiphilic polymers suitable for forming and coating medical devices and which control in situ drug release. The polymers of the present invention have polyester and polyether backbones and are comprised of hydrophilic and hydrophobic monomers including, but not limited to, ε-caprolactone, polyethylene glycol (PEG), trimethylene carbonate, lactide, and their derivatives.

Structural integrity and mechanical durability are provided through the incorporation of monomers such as, but not limited to, lactide. Elasticity and hydrophobicity is provided from monomers comprising caprolactone and trimethylene carbonate. Incorporation of PEG monomers provides a hydrophilic characteristic to the resulting polymer. The amphiphilic polymers of the present invention provide offer a hydrophobic or hydrophilic drug loading capability. Moreover, the polymer can be made biodegradable.

Varying the monomer ratios allows the skilled artisan to fine tune, or to modify, the properties of the polymer. The properties of biodegradable biocompatible amphiphilic polymers arise from the monomers used and the reaction conditions employed in their synthesis including but not limited to, temperature, solvents, reaction time and catalyst choice.

The present invention also takes into account fine tuning, or modifying, the glass transition temperature (Tg) of the biodegradable biocompatible amphiphilic polymers. Drug elution from polymers depends on many factors including density, the drug to be eluted, molecular composition of the polymer and Tg. Higher Tgs, for example temperatures above 40° C., result in more brittle polymers while lower Tgs, e.g lower than 40° C., result in more pliable and elastic polymers at higher temperatures. Drug elution is slow from polymers that have high Tgs while faster rates of drug elution are observed with polymers possessing low Tgs. In one embodiment of the present invention, the Tg of the polymer is selected to be lower than 37° C.

In one embodiment, the polymers of the present invention can be used to form and coat medical devices. Coating polymers having relatively high Tgs can result in medical devices with unsuitable drug eluting properties as well as unwanted brittleness. In the cases of polymer-coated vascular stents, a relatively low Tg in the coating polymer effects the deployment of the vascular stent. For example, polymer coatings with low Tgs are “sticky” and adhere to the balloon used to expand the vascular stent during deployment, causing problems with the deployment of the stent. Low Tg polymers, however, have beneficial features in that polymers having low Tgs are more elastic at a given temperature than polymers having higher Tgs. Expanding and contracting a polymer-coated vascular stent mechanically stresses the coating. If the coating is too brittle, i.e. has a relatively high Tg, then fractures may result in the coating possibly rendering the coating inoperable. If the coating is elastic, i.e has a relatively low Tg, then the stresses experienced by the coating are less likely to mechanically alter the structural integrity of the coating. Therefore, the Tgs of the polymers of the present invention can be fine tuned for appropriate coating applications by a combination of monomer composition and synthesis conditions. The polymers of the present invention are engineered to have adjustable physical properties enabling the practitioner to choose the appropriate polymer for the function desired.

In order to tune, or modify, the polymers of the present invention, a variety of properties are considered including, but not limited to, Tg, connectivity, molecular weight and thermal properties.

In the present invention, the balance between the hydrophobic and hydrophilic properties in the biodegradable biocompatible amphiphilic polymer is controlled. Drug-eluting properties of the biodegradable biocompatible amphiphilic polymers can be tailored to a wide range of drugs. For example, increasing the hydrophobic nature of the polymer increases the polymer's compatibility with hydrophobic drugs. In the case where medical devices coated with polymers of the present invention is desired, the polymers can be tailored to adhere to the particular medical device. In one embodiment of the invention, polyethylene glycol (PEG) is employed for its hydrophilic properties to impart a hydrophilic nature to the polymer. A wide range of PEGs are used wherein Mₙ ranges from about 100 to about 4000. PEGs are not biodegradable; however, if their molecular weight is below 4000, they can be absorbed by giant cell or be excreted by the kidney and other organs. If more hydrophilic components are desired, coupling chemistry can be used to form a polymer having a more hydrophilic nature.

The biodegradable polymers used to form the coatings and implantable medical devices of the present invention can generally be described as follows:

In one embodiment of the present invention, amphiphilic polymers having monomers selected from the group consisting of trimethylene carbonate, polyethylene glycol and lactide are prepared. These monomers are polymerized in the presence of a catalyst including, but not limited to, tin(II)-ethylhexanoate. An exemplary polymer produced with these monomers has the composition of Formula 5:

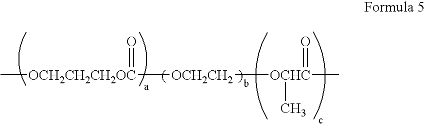

The polyethylene glycol units in Formula 5 provide hydrophilic properties, while the lactic acid and trimethylene carbonate units in the polymer provide elastic and hydrophobic properties. For the polymer of Formula 5, a is an integer from 1 to about 20,000; b is an integer from about 1 to about 100; c is an integer from about 1 to about 20,000 and the sum of a, b and c is at least 4. With control over the variation in a, b and c, the practitioner is able to tune the physical properties of the biodegradable biocompatible amphiphilic polymers.

In another embodiment of the present invention, amphiphilic polymers having monomers selected from the group consisting of ε-caprolactone, polyethylene glycol and lactide are prepared. An exemplary polymer produced with these monomers has the composition of Formula 6:

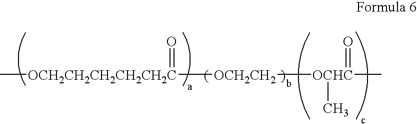

The poly ethylene glycol units in Formula 6 provide hydrophilic properties, while the lactic acid and ε-caprolactone units in the polymer provide elastic and hydrophobic properties. For the polymer of Formula 6, a is an integer from 1 to about 20,000; b is an integer from about 1 to about 100; c is an integer from about 1 to about 20,000 and the sum of a, b and c is at least 4.

In another embodiment of the present invention, the polymer of Formula 5 is reacted with poly(ethylene glycol) bis(carboxymethyl) ether (Formula 7) in the presence of acid to yield the polymer of Formula 8. In Formula 7 and Formula 8, n is an integer from about 1 to about 100.

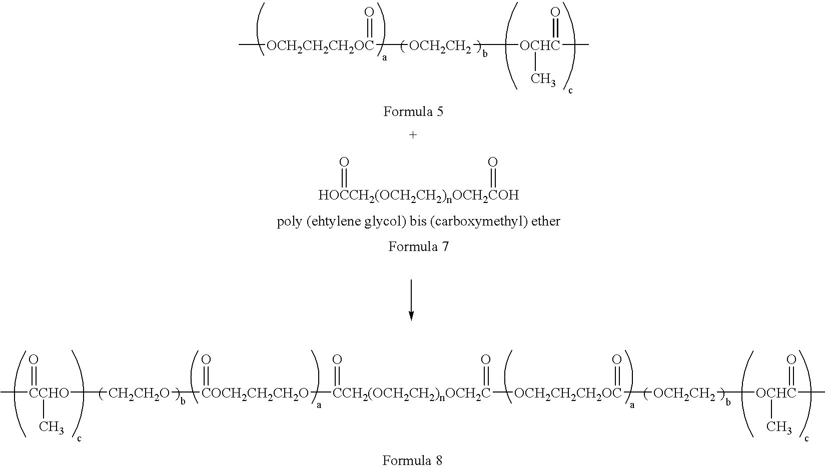

For the polymer of Formula 8, a is an integer from 1 to about 20,000; b is an integer from about 1 to about 100; c is an integer from about 1 to about 20,000 and the sum of a, b and c is at least 4; n can be same or different from b, it is an integer from about 2 to about 100.

In still another embodiment of the present invention, the polymer of Formula 6 is reacted with poly(ethylene glycol) bis(carboxymethyl) ether (Formula 7) in the presence of acid to yield the polymer of Formula 9. In Formula 9, n is an integer from about 1 to about 100.

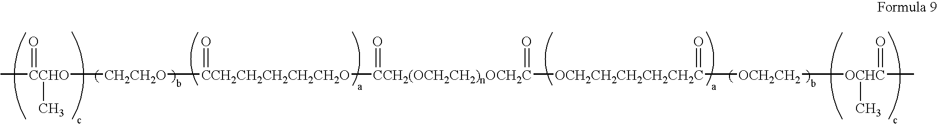

By incorporating poly(ethylene glycol) bis(carboxymethyl) ether into the polymer of Formula 9 the hydrophilic nature of the polymer is enhanced. In this particular embodiment of the polymers of the present invention, integrating additional polyethylene glycol units in the polymer allows fine tuning of the hydrophilic nature of the polymer.

Physical properties of the polymers in the present invention can be fine tuned so that the polymers can optimally perform for their intended use. Properties that can be fine tuned, without limitation, include Tg, molecular weight (both Mₙ and M_{w}), polydispersity index (PDI, the quotient of M_{w}/Mₙ), degree of elasticity and degree of amphiphlicity. In one embodiment of the present invention, the Tg of the polymers range from about −10° C. to about 85° C. In still another embodiment of the present invention, the PDI of the polymers range from about 1.35 to about 4. In another embodiment of the present invention, the Tg of the polymers ranges form about 0° C. to about 40° C. In still another embodiment of the present invention, the PDI of the polymers range from about 1.5 to about 2.5.

The polymers of the present invention, therefore, can be used to form and to coat implantable medical devices. The polymers of the present invention are also useful for the delivery and controlled release of drugs. Drug that are suitable for release from the polymers of the present invention include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

In one embodiment of the present invention, the drug is covalently bonded to a biodegradable biocompatible amphiphilic polymer. The covalently-bound drug is released in situ from the biodegrading polymer with the polymer degradation products thereby ensuring a controlled drug supply throughout the degradation course. The drug is released to the treatment site as the polymeric material is exposed through biodegradation.

Coating implantable medical devices with biodegradable biocompatible amphiphilic polymers that also control drug release is therapeutically advantageous to the patient. Post surgical complications involving medical device implants, e.g. vascular stents, are frequent. Administering drugs combating thrombosis, for example, is a common practice after surgical procedures, especially after cardiothoracic interventions. Drug releasing polymeric coatings on implanted medical devices can offset post surgical side effects by delivering therapeutic agents, such as drugs, directly to the affected areas.

Implantable medical devices suitable for coating with the amphiphilic polymers of the present invention include, but are not limited to, vascular stents, stent grafts, urethral stents, bile duct stents, catheters, guide wires, pacemaker leads, bone screws, sutures and prosthetic heart valves. The polymers of the present invention are suitable for coating and manufacturing implantable medical devices. Medical devices which can be manufactured from the amphiphilic polymers of the present invention include, but are not limited to, vascular stents, stent grafts, urethral stents, bile duct stents, catheters, guide wires, pacemaker leads, bone screws, sutures and prosthetic heart valves.

The controlled release polymer coatings of the present invention can be applied to medical device surfaces, either primed or bare, in any manner known to those skilled in the art. Applications methods compatible with the present invention include, but are not limited to, spray coating, electrostatic spray coating, plasma coating, dip coating, spin coating and electrochemical coating.

The methods described are also useful for coating implantable medical devices only a portion of the medical device such that the medical device contains portions that provide the beneficial effects of the coating and portions that are uncoated. The coating steps can be repeated or the methods combined to provide a plurality of layers of the same coating or a different coating. In one embodiment, each layer of coating comprises a different polymer or the same polymer. In another embodiment each layer comprises the same drug or a different drug.

In one embodiment of the present invention, an amphiphilic polymer of the present invention is chosen for a particular use based upon its physical properties. For example, a polymer coating provides additional structural support to a medical device by increasing the content of lactic acid in the polymer. In still another embodiment, a polymer coating on a medical device decreases friction between the medical device and the surrounding tissue, or between the medical device and the delivery system, facilitating the implantation procedure.

Recently, the medical community has increased its reliance on implantable medical devices manufactured from biocompatible polymers. The biodegradable biocompatible amphiphilic polymers of the present invention are particularly suitable for manufacturing implantable medical devices since the methods and compositions disclosed herein allow the fine tuning of the structural properties of the polymers by using various ratios of monomers in the synthesis of the polymers.

In one embodiment of the present invention, a vascular stent is manufactured from the biodegradable biocompatible amphiphilic polymers of the present invention. The advantages of the biodegradable biocompatible amphiphilic polymer coating also apply to vascular stents manufactured from biodegradable biocompatible amphiphilic polymers.

The biodegradable biocompatible amphiphilic polymers described herein can be tuned to biodegrade at various lengths of time by varying the monomer composition of the polymer. An exemplary polymer synthesized with polyethylene glycol monomers will be more hydrophilic than polymers without PEG monomers and therefore will have slower degradation times.

### EXAMPLES

The following non limiting examples provide methods for the synthesis of exemplary polymers according to the teachings of the present invention.

### Example 1

### Synthesis of a Polymer of Formula 5

To a reaction vessel is added polyethylene glycol (PEG) with molecular weight of about 3500 (1.3 g, about 0.4 mmol), trimethylene carbonate (15 g, 150 mmol), dl lactide (35 g, 243 mmol) and tin(II)2-ethylhexanoate (0.05 g, 0.1 mmol). The vessel is purged with nitrogen gas. The mixture is heated (150° C.) and stirred (320 rpm) for 24 hours then cooled to ambient temperature. The polymer is discharged and dissolved in chloroform (2000 mL). Methanol (500 mL) is added precipitating the polymer from solution. The solution is filtered and the mother liquor disregarded. The solid polymers are then re-dissolved in chloroform and poured into Teflon trays.

### Example 2

### Synthesis of a Polymer of Formula 8

To a reaction vessel is added polyethylene glycol (PEG) with molecular weight of about 3500 (1.3 g, about 0.4 mmol), trimethylene carbonate (15 g, 150 mmol), dl lactide (35 g, 243 mmol) and tin(II)2-ethylhexanoate (0.05 g, 0.1 mmol). The vessel is purged with nitrogen gas. The mixture is heated (150° C.) and stirred (320 rpm) for 24 hours. Poly(ethylene glycol)-bis-(carboxymethyl) ether (0.5 g, 0.6 mmol) is added and a vacuum is applied, the mixture is stirred for an additional 4 hours and cooled to ambient temperature. The polymer is discharged and dissolved in chloroform (2000 mL). Methanol (500 mL) is added precipitating the polymer from solution. The solution is filtered and the mother liquor discarded. The solid polymers are then re-dissolved in chloroform and poured into Teflon trays.

### Example 3

### Manufacturing Implantable Vascular Stents

The present invention pertains to biodegradable biocompatible amphiphilic polymers used for the manufacture of medical devices and medical devices coatings. The biodegradable biocompatible amphiphilic polymers disclosed in the present invention retain and release bioactive drugs. Example 3 discloses a non-limiting method for manufacturing stents made of biodegradable biocompatible amphiphilic polymers according to the teachings of the present invention.

For exemplary, non-limiting, purposes a vascular stent will be described. A biodegradable biocompatible amphiphilic polymer is heated until molten in the barrel of an injection molding machine and forced into a stent mold under pressure. After the molded polymer (which now resembles and is a stent) is cooled and solidified the stent is removed from the mold. In one embodiment of the present invention the stent is a tubular shaped member having first and second ends and a walled surface disposed between the first and second ends. The walls are composed of extruded polymer monofilaments woven into a braid-like embodiment. In the second embodiment, the stent is injection molded or extruded. Fenestrations are molded, laser cut, die cut, or machined in the wall of the tube. In the braided stent embodiment monofilaments are fabricated from polymer materials that have been pelletized then dried. The dried polymer pellets are then extruded forming a coarse monofilament which is quenched. The extruded, quenched, crude monofilament is then drawn into a final monofilament with an average diameter from approximately 0.01 mm to 0.6 mm, preferably between approximately 0.05 mm and 0.15 mm. Approximately 10 to approximately 50 of the final monofilaments are then woven in a plaited fashion with a braid angle about 90 to 170 degrees on a braid mandrel sized appropriately for the application. The plaited stent is then removed from the braid mandrel and disposed onto an annealing mandrel having an outer diameter of equal to or less than the braid mandrel diameter and annealed at a temperature between about the polymer glass transition temperature and the melting temperature of the polymer blend for a time period between about five minutes and about 18 hours in air, an inert atmosphere or under vacuum. The stent is then allowed to cool and is then cut.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A biodegradable biocompatible amphiphilic polymer comprising:
a polyester and polyether backbone; and wherein said polymer comprises at least two polymerizable monomers selected from the group consisting of trimethylene carbonate, lactide, ε-caprolactone, polyethylene glycol, glycolide, 4-tert-butyl caprolactone, N-acetyl caprolactone, poly(ethylene glycol) bis(carboxymethyl) ether as depicted in Formula 7 wherein n ranges from about 1 to about 100 and combinations thereof.

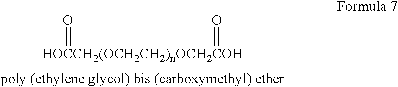

2. The biodegradable biocompatible amphiphilic polymer of claim 1 wherein said polymer is used to coat implantable medical devices.

3. The biodegradable biocompatible amphiphilic polymer of claim 1 wherein said polymer is used to form an implantable medical device.

4. The biodegradable biocompatible amphiphilic polymer of claim 1 wherein said polymer further comprises a drug.

5. The biodegradable biocompatible amphiphilic polymer of claim 1 wherein said polymer comprises the structure of Formula 5: 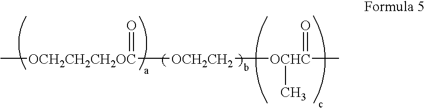
and wherein a is an integer from 1 to about 20,000; b is an integer from about 1 to about 100 and c is an integer from about 1 to about 20,000 and the sum of a, b and c is at least 4.

6. The biodegradable biocompatible amphiphilic polymer of claim 5 wherein said polymer comprises the structure of Formula 5: 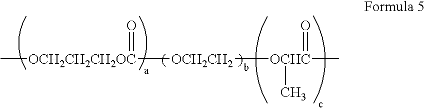
and wherein a is an integer from about 4 to about 25; b is an integer from about 1 to about 3 and c is an integer from about 10 to about 40.

7. The biodegradable biocompatible amphiphilic polymer of claim 1 wherein said polymer comprises the structure of Formula 6: 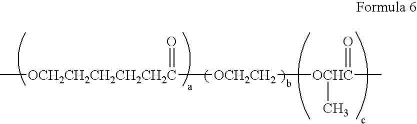
and wherein a is an integer from 1 to about 20,000; b is an integer from about 2 to about 100, and c is an integer from about 1 to about 20,000 and the sum of a, b and c is at least 4.

8. The biodegradable biocompatible amphiphilic polymer of claim 1 wherein said polymer comprises the structure of Formula 8; 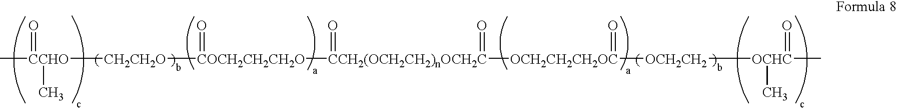
and wherein a is an integer from 1 to about 20,000; b is an integer from about 1 to about 100; c is an integer from about 1 to about 20,000; the sum of a, b and c is at least 4 and n is an integer from about 1 to about 100.

9. The biodegradable biocompatible amphiphilic polymer of claim 1 wherein said polymer comprises the structure of Formula 9; 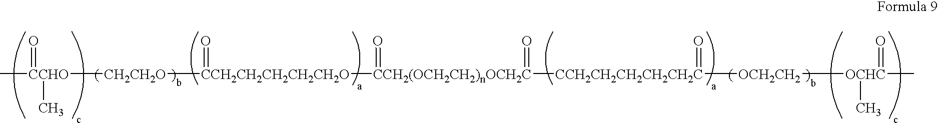
and wherein a is an integer from 1 to about 20,000; b is an integer from about 2 to about 100; c is an integer from about 1 to about 20,000; the sum of a, b and c is at least 4 and n is an integer form about 1 to about 100.

10. The biodegradable biocompatible amphiphilic polymer of claim 1 wherein the polydispersity index is between about 1.35 and about 6.

11. The biodegradable biocompatible amphiphilic polymer of claim 10 wherein the polydispersity index is between about 2 and about 4.

12. The biodegradable biocompatible amphiphilic polymer of claim 1 wherein the glass transition temperature is between about −70° C. and about 85° C.

13. The biodegradable biocompatible amphiphilic polymer of claim 12 wherein the glass transition temperature is between about −60° C. and about 70° C.

14. The biodegradable biocompatible amphiphilic polymer of either of claims 2 or 3 wherein said implantable medical device is selected from the group consisting of vascular stents, shunts, vascular grafts, stent grafts, heart valves, catheters, pacemakers, pacemaker leads, bile duct stents and defibrillators.

15. A coating for an implantable medical device comprising:
a biodegradable biocompatible amphiphilic polymer comprising a polyester and polyether backbone; and wherein said polymer comprises at least two polymerizable monomers selected from the group consisting of trimethylene carbonate, lactide, ε-caprolactone, polyethylene glycol, glycolide, 4-tert-butyl caprolactone, N-acetyl caprolactone and poly(ethylene glycol) bis(carboxymethyl) ether as depicted in Formula 7 wherein n ranges from about 1 to about 100 and combinations thereof.

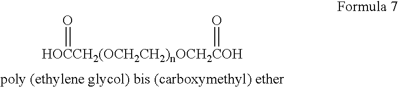

16. The implantable medical device of claim 15 wherein said medical device is selected from the group consisting essentially of, vascular stents, shunts, vascular grafts, stent grafts, heart valves, catheters, pacemakers, pacemaker leads, bile duct stents and defibrillators.

17. An implantable medical device comprising:
a biodegradable biocompatible amphiphilic polymer comprising a polyester and polyether backbone; and
wherein said polymer comprises two or more polymerizable monomers selected from the group consisting of trimethylene carbonate, lactide, ε-caprolactone, polyethylene glycol, glycolide, 4-tert-butyl caprolactone, N-acetyl caprolactone and poly (ethylene glycol) bis(carboxymethyl) ether as depicted in Formula 7 wherein n ranges from about 1 to about 100.

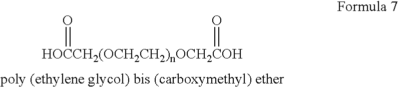

18. The implantable medical device of claim 17 wherein said medical device is selected from the group consisting essentially of, vascular stents, shunts, vascular grafts, stent grafts, heart valves, catheters, pacemakers, pacemaker leads, bile duct stents and defibrillators.
